(19) United States
(12) Patent Application Publication
(10) Pub. No.: US 2010/0086918 A1
(43) Pub. Date: Apr. 8, 2010

(54) Methods for High Sensitivity Detection of Genetic Polymorphisms

(75) Inventors: Dennis A. Carson, La Jolla, CA (US); Yu-Tsueng Liu, San Diego, CA (US)

Correspondence Address: DLA PIPER LLP (US), 4365 EXECUTIVE DRIVE, SUITE 1100, SAN DIEGO, CA, 92121-2133, US

(73) Assignee: THE REGENTS OF THE UNIVERSITY OF CALIFORNIA, Oakland, CA (US)

(21) Appl. No.: 12/375,912

(22) PCT Filed: Jul. 31, 2007
(86) PCT No.: PCT/US07/17128

§ 371 Date: Nov. 17, 2009

Related U.S. Application Data
(60) Provisional application No. 60/834,684, filed on Jul. 31, 2006.

(51) Int. Cl. C12Q 1/68; C07H 21/04

(52) U.S. Cl. 435/6; 536/23.1

(57) ABSTRACT

Multiplex PCR-based methods for detecting a variant polynucleotide having a nucleotide sequence differing from the wild-type nucleotide sequence of a nucleic acid molecule, wherein the variant polynucleotide is in a sample containing an excess of the wild-type nucleic acid molecule. The methods are particularly useful for detection of deletions from, or translocations and inversions in, genomic DNA. The susceptibility to, diagnosis of, and progression of a disease clinically related to the occurrence of such polymorphisms in an individual may also be confirmed and monitored using the multiplex PCR-based methods or by detecting RNA fusion transcripts in a sample that correspond to previously identified deletions, translocations or inversions in genomic DNA.

### FIELD OF THE INVENTION

The invention relates to assay methods for identification of polymorphisms in genes, especially deletions or translocations in genomic DNA. The invention further relates to identification of chromosomal anomalies arising from such polymorphisms in mammalian cells.

### BACKGROUND OF THE INVENTION

Sequence variations in genomic DNA from wild-type, such as gene deletions, are often associated with the onset and progression of primary cancers. For example, deletions of the CDKN2A gene coding for the p16^{INK4a} and p14^{ARF} proteins commonly occur in human cancer cell lines. However, the size of the deletions, and therefore the location of their breakpoints, vary widely.

The diagnostic and cancer monitoring potential of deletions from genomic DNA has been difficult to exploit clinically because (a) tumor specimens are invariably contaminated with normal cells, demanding time consuming methods for tumor nucleic acid extraction, and (b) the sizes of deletions in particular can vary from <1 to >40 kb. Currently available methods for deletion mapping (including Southern blotting, LOH analysis, fluorescence in situ hybridization, real time PCR and array based comparative genome hybridization [CGH]) all suffer from various technical limitations and, consequently are not able to detect many deletions, nor to precisely characterize them.

If a protein product of a gene is ubiquitously expressed, immunohistochemical (IHC) detection of the protein can be used as a screening surrogate for genetic or epigenetic gene inactivation. However, the production of many cancer-related proteins, such as p15^{INK4b}, p16^{INK4a}, and p14^{ARF}, varies with cell differentiation, growth and senescence. Further, where the cancer-related sequence (in genomic DNA or a fusion transcript) is not known in advance, existing detection protocols require the gene sample to be substantially (e.g., 80%) pure. Accordingly, IHC and other existing techniques for analysis of these proteins has not been an accurate screen for assessing deletions or sequence variations in the coding gene (e.g., for p16/p14 and p15).

A need, therefore, exists for a method that will enable detection of even small gene deletions in the presence of a vast excess of wild-type gene (e.g., from non-isolated primary tumors). With such a method in hand, breakpoint-specific molecular probes for use in personalized monitoring of cancer progression in individuals may be developed.

### SUMMARY OF THE INVENTION

The invention provides a multiplex PCR-based method for detecting a polynucleotide having a nucleotide sequence differing from the wild-type polynucleotide sequence of a gene, wherein the variation is a deletion, translocation, inversion or fusion of nucleotides, and the variant polynucleotide is in the presence of an excess of the wild-type molecule. More specifically, the detection is of a polymorphism in genomic DNA, and is accomplished directly and/or may be confirmed by detection of a corresponding abnormal RNA fusion transcript.

The multiplex PCR method is particularly advantageous in that it allows for identification and characterization of deletion segments and their breakpoint boundaries, even against a background containing a vast excess of the wild-type molecule; e.g., at least a predominance (>50%) of wild-type and beyond the limits of conventional assays, such as IHC (e.g., ≧80%). For example, the method allows for detection of chromosomal gene deletions and mapping their breakpoints in samples of genomic DNA containing up to about 99.9% wild-type DNA contamination. In another embodiment, the presence of an abnormal RNA fusion transcript in a sample was detected in a sample containing ˜3000 times wild-type RNA.

To this end, multiple primer pairs approximating the flanking sequence of a deletion sequence are subjected to multiplex PCR. Each number of the primer pair is spaced about ≧1 kb from the other member, or may be placed closer together in embodiments of the invention utilizing a poison primer. Forward and reverse primer pairs are provided and separated into groups (up to about 100 primers per group) for use in multiplicity of multiplex PCR reactions, comprising a primary amplification step. A secondary amplification step may be performed to increase the product specificity for the boundaries of the sequence variation. To target relatively small (<1 kb) deletions, poison primer PCR using a primer pair external to the variant segment and a third primer internal to the segment will be utilized to target the deleted sequence.

The invention further provides means for determining the susceptibility of an individual organism, such as a mammal (and particularly a human), to develop a disease clinically related to the occurrence of deletions, inversions or translocations in genomic DNA, such as cancer, as well as diagnosing and monitoring the progression of such a disease in an individual by tracking and comparing the occurrence of targeted deletions or translocations in different populations of cells, or in the same population of cells over time. For example, with knowledge of genomic breakpoints or the identity of related fusion transcripts, molecular probes are developed to inform the clinician of the presence of a sequence variation in genomic DNA or RNA transcripts with pathological implications for the onset and progression of cancer.

To this end, a first embodiment of the invention provides multiple primer pairs that are hybridizable to a target polynucleotide (e.g., one or more chromosomal gene segments), where each number of the primer pair is spaced≧about 1 kb from the other member. Forward and reverse primer pairs are provided and separated into groups (up to about 100 primers per group) for use in multiplicity of multiplex PCR reactions, comprising a primary amplification step. Advantageously, the array reactions are suitable to automation by separation of the primer pair groups into wells of a microtiter plate.

Of the multiplex PCR products, few (generally, one or two) will span the deletion, translocation or inversion boundary, since the other primer pairs should be spaced too far from the boundary for efficient amplification.

Preferably, the primary boundary-spanning amplification products (amplicons) will be further amplified to increase the product specificity for the target boundaries. Nested PCR methods are particularly useful for use in this secondary amplification step. To target relatively small (<1 kb) deletions, poison primer PCR using a primer pair external to the targeted deletion segment and a third primer internal to the segment will be utilized to target the deletion in the wild-type genome.

To characterize the boundaries of a genomic deletion, translocation or inversion, or to identify an abnormal fusion transcript, sequence analysis is performed on the amplicons obtained from the primary or, if performed, secondary amplification steps. In one embodiment of the invention, the analysis step is performed on a genomic tiling array. According to this embodiment, amplicons indicative of the boundaries of a sequence variation, such as deletion breakpoints, obtained from the PCR step(s) of the invention are labeled for hybridization on one or more gene-specific tiling arrays. The boundaries of the targeted sequence variation are considered confirmed by probe hybridization to the putative breakpoints identified in the PCR step(s).

In an alternative embodiment of the invention, the sequence variation is characterized by direct sequencing according to conventional techniques.

In a further alternative embodiment of the invention, the primer pair groups prepared for multiplex PCR are not separated physically into wells before amplification. Instead, the primer pairs and template polynucleotides are separated into groups by admixture in a water-in-oil emulsion. Most preferably, the primers are bound to a solid phase support, such as nanoparticles prior to admixture into the water-in-oil emulsion. Amplification may therefore be performed in a single tube rather than a multiwell plate.

In an additional variation on the invention, probes specific to one or more sequence variations detected according to the assays of the invention are developed. Such probes allow for determining the susceptibility of an individual to develop a disease clinically related to the occurrence of genomic deletions, translocations or inversion, such as occur in certain cancers or heart disease conditions. The methods of the invention also permit diagnosis and monitoring of the progression of such a disease; e.g., as measured by changes in the length, size or number of polymorphisms in the target nucleic acid, especially genomic DNA.

### BRIEF DESCRIPTION OF THE DRAWINGS

FIG. 1. The CDKN2A/MTAP genome region. The genomic map covers 500 kb around CDKN2A according to Ensemble™ version 36. The MTAP transcript is generated by the forward strand, while CDKN2A/B are encoded by the reverse strand.

FIG. 2. Overall screening strategy illustrating by detection of CDKN2A breakpoints. MTAP staining divides samples into two classes, for identifying deletions in the proposed 500 kb genomic region. Multiplex PCR with sets of primers spread across the region, combined with genomic tiling microarray spotted with PCR amplified non-repetitive genomic probes allows for deletion mapping. A predicted result is shown when a deletion is present. The deleted genomic sequence is bordered by two peaks in the diagram. The exact breakpoint can be mapped by various techniques to yield a tumor specific breakpoint signature.

FIG. 3. Primer Approximation Multiplex PCR (PAMP). Primers to amplify genomic sequences around the CDKN2A locus can be divided into 20 groups: 10 each for forward (F1-F10) and reverse (R1-R10) groups (A). Multiplex PCR reactions are set and represented as a matrix to include one forward and one reverse primer group. The expected PCR results are shown as gray scale shadows in the matrix (B). This schematic shows that only group pairs close to the breakpoint give PCR products (F-3-R3, F3-R4, F4-R3).

FIG. 4. Breakpoint identification by PAMP with a minigenomic tiling array (A) Four groups of primers near the potential breakpoints were generated for PAMP. The mapped CDKN2A breakpoints of the Detroit 562 cell line in this and other studies are indicated. The “e1” and “e2” designations are the relative positions of INK4A exons 1 and 2. The tiling probes for the array are indicated. (B) The same sets of primers were used for PCR reactions on Detroit 562 (Mut) and HEK293 (WT) cells for CDKN2A breakpoint detection. The amplicons were labeled with different dyes, yielding a green signal for variant samples and a red signal for WT samples. The first row of the minigenomic array was spotted with the probes shown in panel A. Cot-1 DNA spots are indicated. The rest of the spots are herring sperm DNA.

FIG. 5. For comparison, identification of the CDKN2A breakpoint in Detroit 562 by singleplex nested PCR. (A) Ratio intensity vs. probe genomic location of Detroit 562/HEK293 samples on an INK4A minigenomic tiling array. The breakpoint site was mapped using a nested PCR strategy with a common external primer and two different internal primers (B and C). A total of 0.1 μg of genomic DNA was used as template with various ratios (2-100%) of DNA from Detroit 562 (CDKN2A deleted) mixed with DNA from HEK293 (intact CDKN2A locus). “0” represents no input template DNA (water control). The amplicons were gel purified and sequenced, which confirmed the breakpoint created by the 14 kb deletion (D).

FIG. 6. Breakpoint identification by PAMP with a water-in-oil emulsion. A schematic for use of the primers in the water-in-oil emulsion compartments is illustrated. Every group of primers is entrapped in wax nanoparticles (about 100 nm in diameter), which melt when the temperature is higher than 55° C. The nanoparticles are diluted such that less than 3 particles are encapsulated in a droplet during water-in-oil emulsification with other components for PCR (templates, enzyme, nucleotides and buffer). The wax nanoparticles are melted when the PCR program starts with 95° C. and subsequent thermocycling. This design allows for PCR reactions to be assembled in a single tube. It also increases PCR specificity through a hot start design with wax entrapped primers.

FIG. 7. A schematic illustrating the “poison primer” technique for the secondary amplification step of the invention.

FIG. 8. TMPRSS2:ERG Exon Mapping Strategy. (A) The RT-PCR is primed with 3′ primer at exon 6 of the ERG and 5′ primer at exon 1 of the TMPRSS2. Only fusion transcript can be exponentially amplified since the two primers are at different genes. The probes on the array are derived from exons 1-3 of the TMPRSS2 and exons 1-5 of the ERG. (B) The hybridization pattern of RT-PCR labeled amplicons with total RNA derived from VCaP cell line. The result clearly shows the fusion junction is at exon 1 of the TMPRSS2 and exon 4 of the ERG as illustrated for the fusion scenario in panel A. A probe that spans on the junction of exon 1 and 2 of the TMPRSS2 is labeled as “½”.

FIG. 9. Assay Sensitivity. VCaP total RNA was serially diluted in solution containing HeLa RNA to mimic the heterogeneous cell population in primary tumor or human body fluids. The total amount of RNA for each reaction is 100 ng. The intensity of the each expected feature (T1, G4, G5) is at the saturated level. The signal disappeared when the VCaP RNA was diluted from 1:3125 (32 pg) to 1:15625 (6.4 pg).

### DETAILED DESCRIPTION OF THE INVENTION

In its broadest sense, the present invention allows the detection and characterization of any polymorphism in, or deletion of, a target nucleic acid sequence of diagnostic or therapeutic relevance, where the target nucleic acid sequence is present in a biological cell sample from any organism, such as the margins of a primary tumor or a regional lymph node. Thus, the target nucleotide sequence may be, for example, deletions, translocations or inversions in genomic DNA, or exon junctions in corresponding fusion RNA transcripts. Further, the method of the invention can be used to detect and characterize multiple target polynucleotides; e.g., multiple deletion segments.

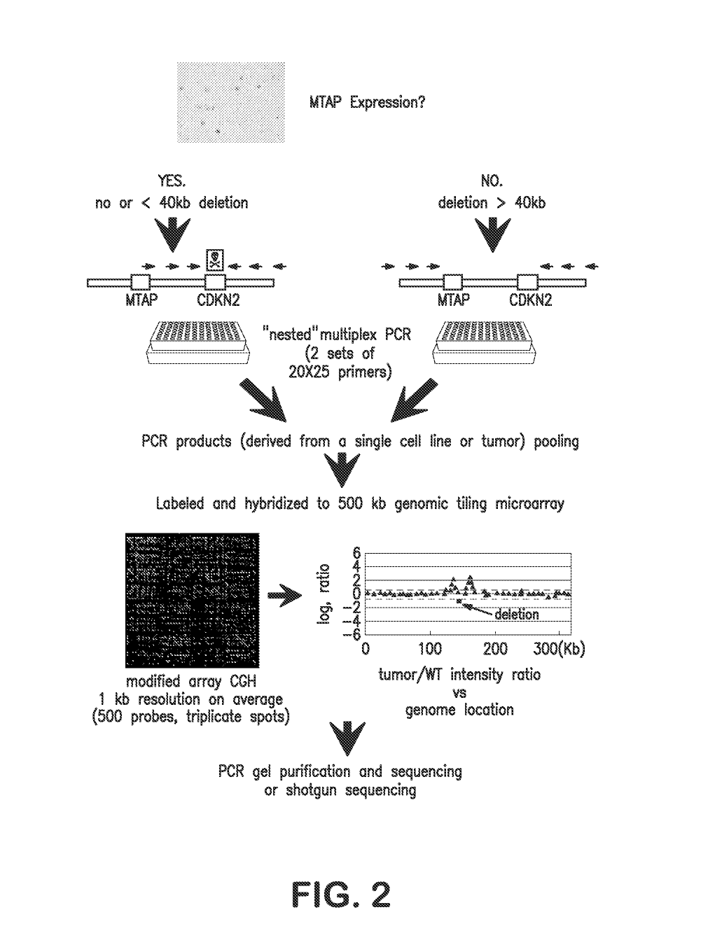

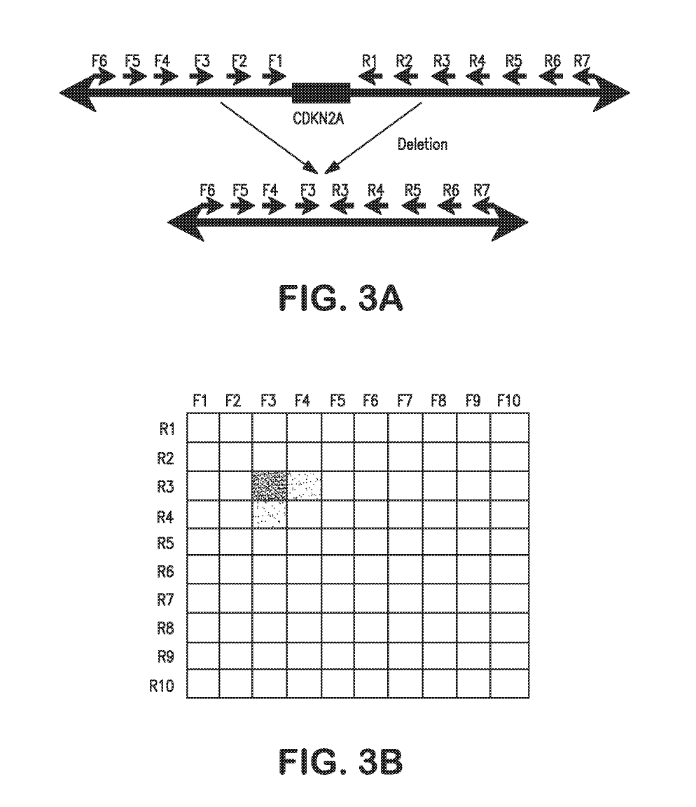

With respect to deletion segments in particular, the invention exploits the fact that a nucleic acid sequence from which a polynucleotide segment has been deleted is shorter than, and therefore should be preferentially amplified compared to, the longer wild-type sequence using “approximated” flanking primers (FIGS. 2 and 3). For ease of reference, the PCR aspects of the inventive methods shall be referred to herein as “Primer Approximation/Multiplex PCR,” or “PAMP”.

I. Overview of PAMP Methodology.

The invention adapts and utilizes techniques for PCR amplification of DNA in a biological sample. The basic techniques for performing PCR are well-known in the art. For further details, consult U.S. Pat. Nos. 4,683,195 and 4,683,202 to Mullis, et al., the disclosures of which are incorporated herein.

Primer approximation PCR techniques have been previously used to isolate deletion variants in C. elegans (Jansen, et al, Nat. Genetics, 17:119-121, 1997). The method relies on identifying a single band that is the product of a successful PCR when a pair of specific primers is brought together by deletion, on an agarose gel. However, the procedure can yield ambiguous results since only deletions near single primer pairs can be identified.

Multiplex PCR enables generation of multiple amplicons in a single PCR reaction, and is especially useful in amplifying nucleic acids of known sequences (see, e.g., Boriskin, et al., J. Clin. Microbiol., 42: 5811-5818, 2004). The existing method is not especially useful, however, for detecting deletion segments of unknown sequence or length.

The Jansen, et al. and Boriskin, et al. papers are incorporated herein by this reference to illustrate the general application of the primer approximation and multiplex PCR techniques in the art before the invention.

The PAMP approach of the invention combines, adapts and refines the general principles of primer approximation PCR and multiplex PCR, as illustrated in FIGS. 2 and 3, as well as in Examples I and II. An overall screening strategy accomplished by the invention is depicted in FIG. 2. In the schematic of FIG. 3, evenly spaced primers surrounding the locus (or loci) of interest are divided into 20 groups for multiplex PCR. There are 10 groups each of forward primer mixtures, F1, F2 . . . , F10 and reverse primer mixtures R1, R1, R2, . . . , R10, respectively (FIG. 3A). Therefore, there are 100 pairs (F1-R1, F1-R2, . . . , F1-R10; F2-R1, F2-R2 . . . , F2-R10; . . . ; F10-R1, F10-R2, . . . , F10-R10) of multiplex PCR reactions (FIG. 3B). Each primer approximation reaction (multiplied for multiplex PCR at about 1 kb intervals) relies on the increased efficiency of standard PCR in amplifying shorter fragments. The deletion will bring the two primers closer together if a deletion of genomic DNA including the segments spaced at about 1 kb intervals has occurred. Those of ordinary skill in the art will recognize that shorter probes (e.g., 50-70 bp) should be utilized for analysis of gene sequences with numerous repetitive motifs.

A representative protocol for each primer approximation reaction (multiplied for multiplex PCR) is described in Example II. In general, only one or two pairs of PCR reactions will produce specific PCR products spanning the deletion boundary, since the other primer pairs should be too far from the breakpoint for efficient amplification.

The multiplex PCR conditions may be varied by those of ordinary skill in the art (e.g., according to the number of primers used, the extent of automation, the polymerases applied, and the like), but are essentially as described in Boriskin, et al., J. Clin. Microbiol., 42:5811-5818, 2004, and in Example I, using 96-well plates. Each plate will preferably be used for 1 cell line or tumor sample. Further, although there are multiple possible combinations of the primer groups (100 being the example shown in FIG. 3), the number of primer groups needed can be streamlined by pre-primary amplification screening of the genomic DNA sample using existing, albeit less sensitive techniques; e.g., immunohistochemistry (IHC). Primer selection will preferably be made so as to minimize primer-dimer formation; to this end, a useful technique for primer design is set forth in co-pending and commonly assigned U.S. Provisional Patent Application No. 60/931,793, filed Mar. 25, 2007.

The primary amplification step itself can be rendered semi-automatic by using commercially available robots for liquid handling, addition of reagents and the like, such as the BIOMEX FX™ from Beckman-Coulter. A particular application of the primary multiplex PCR step of the invention is illustrated in Example II.

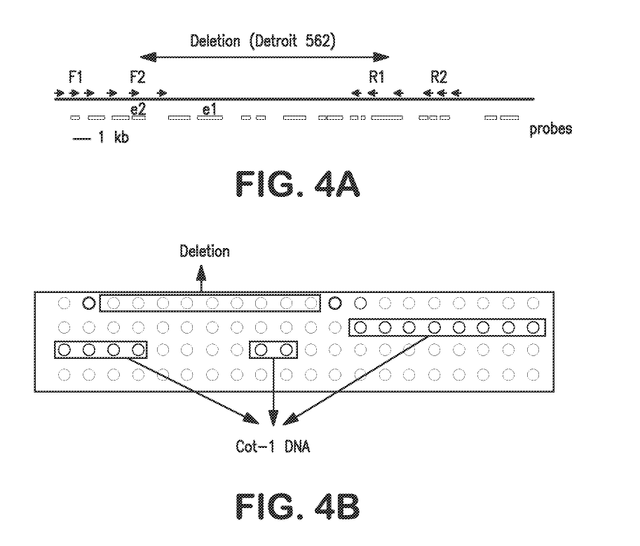

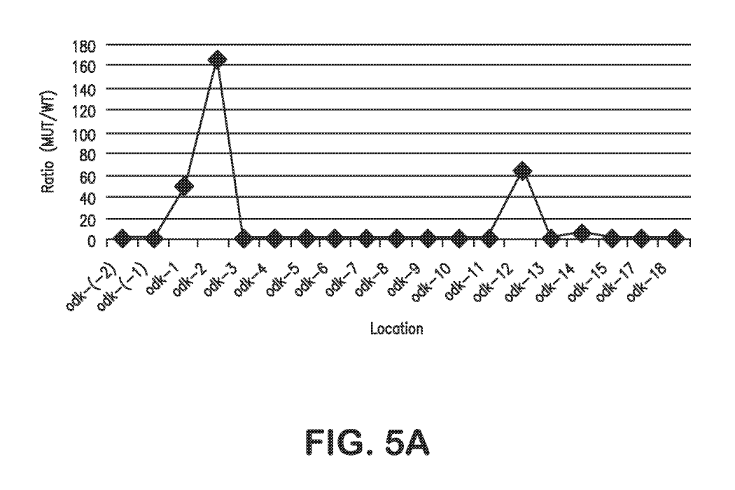

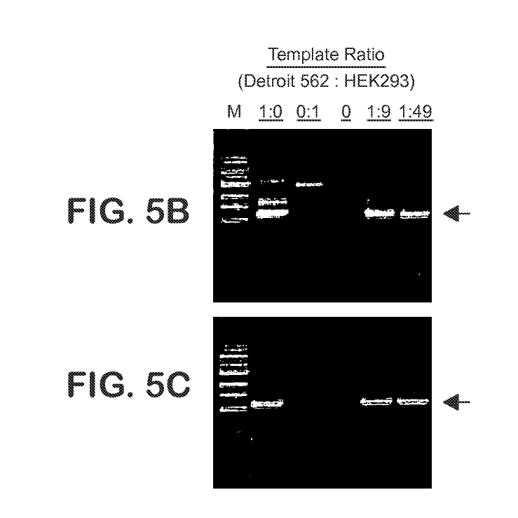

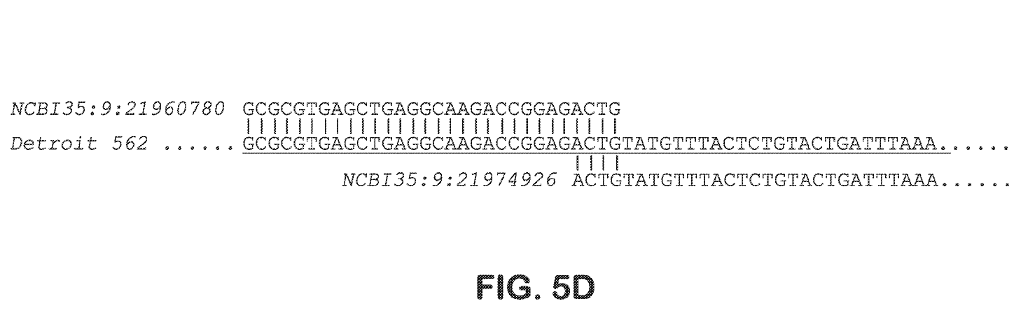

Secondary groups of different primers can be used to increase the specificity and further amplify the products from the first multiplex PCR reactions (equivalent to internal primers for nested PCR). Then aliquots from each PAMP can be mixed to hybridize on a single genomic tiling array. Unlike traditional array CGH, only spots representing genomic sequences near the breakpoints will light up on the array (see, schematic in FIG. 2, CDKNA2 specific results shown in FIGS. 4 and 5). Practiced as disclosed herein, the assay of the invention is sufficiently sensitive to detect a variant molecule in a sample containing a vast excess of the wild-type molecule, wherein a predominance (>50%) of the molecules present in the sample have the wild-type structure. For example, the invention may detect a deletion from genomic DNA in a sample of up to about 99.9% wild-type genomic DNA; i.e., DNA which does not contain the deletion (see, Example VI).

If a secondary amplification is performed to increase the specificity of the amplicons for deletion breakpoints, nested PCR utilizing additional primers hybridizable to the boundaries of the deleted gene segments will be performed (see, e.g., Example II). General techniques for performing nested PCR are well known in the art.

Briefly, nested PCR uses two sets of amplification primers. The target nucleic acid sequence of one set of primers (termed “inner” primers) is located within the target sequence of the second set of primers (termed “outer” primers). In practice, a standard PCR reaction is first run with the patient sample using the “outer primers”. Then a second PCR reaction is run with the “inner primers” using the product of the first reaction as the amplification target. This procedure increases the sensitivity of the assay by reamplifying the product of the first reaction in a second reaction. The specificity of the assay is increased because the inner primers amplify only if the first PCR reaction yielded a specific product.

II. Poison Primer Adaption of PAMP Methodology.

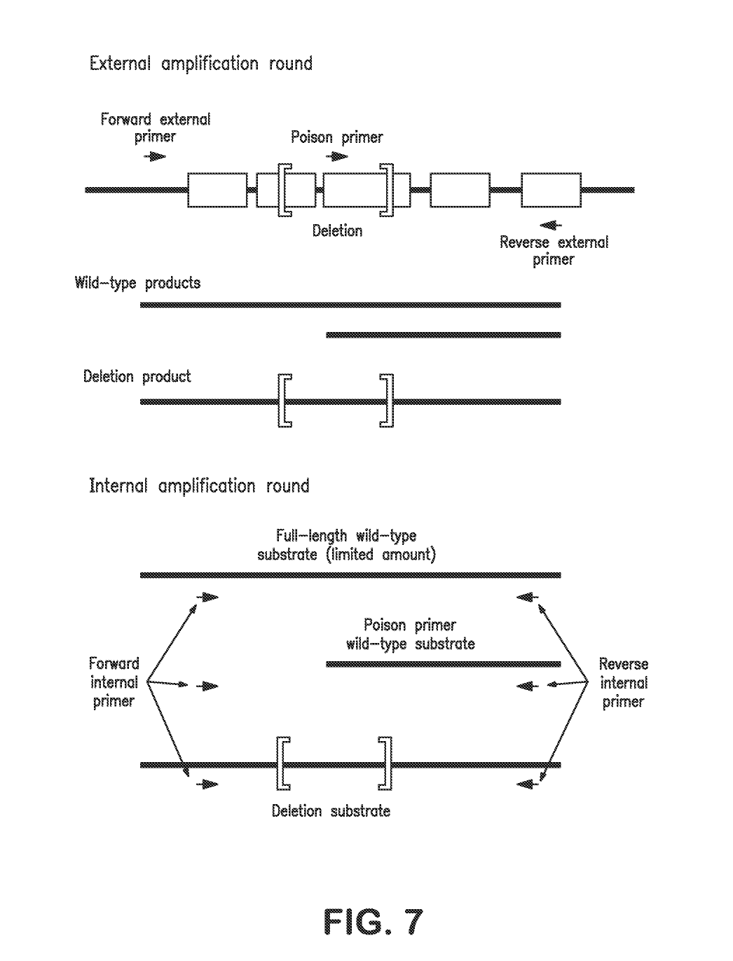

While the PAMP approach works well for larger genomic deletions, it is less discriminating for smaller deletions (i.e., less than approximately 1 kb). However, by employing a “poison primer” nested PCR strategy for the secondary amplification step, it is possible to insure that even very small deletions are selectively amplified (see, e.g. Edgley, et al., Nucleic Acids Res., 30:e52, 2002, incorporated herein by this reference; FIG. 7, and Example V). Such an approach has recently proved useful for C. elegans variant screening and for the detection of small deletions in mutagenized mouse embryonic stem cells (Greber, et al., Hum. Mutat., 25:483-490, 2005). In general, the poison primer strategy utilizes primer pairs hybridizable to a segment of a deleted gene segment during the multiplex PCR step (see, FIG. 7).

The amplicons from smaller deletions are not sufficiently different from wild-type to provide a competitive advantage in PCR. However, when a “poison primer” from the deleted sequence anneals to the wild-type genome, it competes for the amplification of the WT genome with the common set of primers, which amplifies both wild-type and variant genomes. The amplification reactions with poison primers in wild-type DNA are favored, since the PCR products are smaller.

Briefly, a third functional PCR primer that falls between the two external primers flanking a deletion segment identified in the primary amplification step is designed (FIG. 7 and Example V). Amplification from the wild-type template leads to the production of two fragments, one full-length and one relatively short. In practice, the shorter fragment is produced much more efficiently than the longer. Amplification from a variant template, in which the site for the third internal primer is deleted, leads to the production of a single variant fragment from the normal external primers.

In a further round of PCR, two primers are placed just inside the external first round primers. The shorter wild-type band from the first round cannot serve as a template for the second round PCR because it does not include one of the second round primer sites. The longer wild-type fragment can serve as a template, but because its production is limited by competition in the first round, its production in the second round is limited correspondingly. The lower level of effective wild type gives the deletion fragment an advantage since the majority of the primary PCR products are poison primer derived, and lack one of the secondary primer annealing sequences (Example V). The extension of this methodology to a multiplex format yields the PPMP (poison primer [approximation] multiplex PCR) method of the invention.

III. Primer Autopairing Via Emulsification (PAVE).

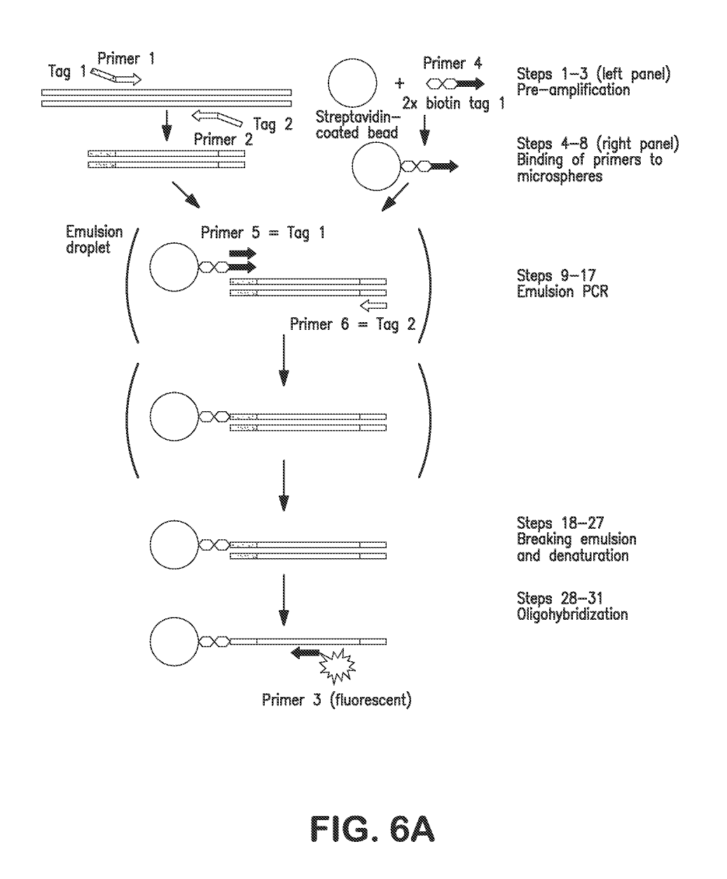

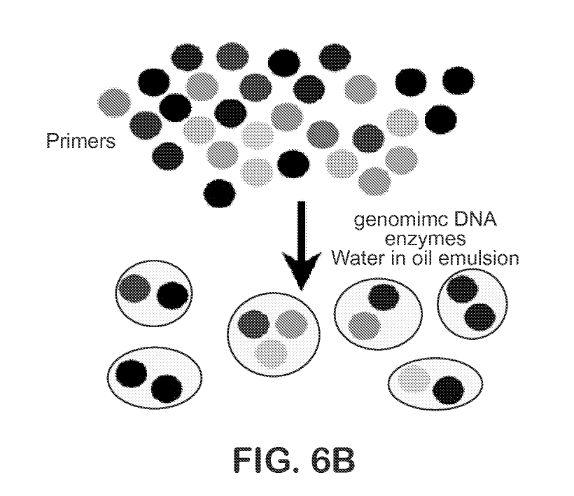

An alternative amplification method in which use of microtiter plates is not required involves compartmentalization of primer pairs, templates and a solid phase support (e.g., microparticles or beads). The approach, known in the art as water-in-oil emulsion PCR is generally described in Diehl, et al., Natl. Methods, 3:551-559, 2006; Kojima, et al., Nucleic Acids Research, 33(17):e150, 2005; and Shendurne, et al., Science, 309:1728-1732, 2005 (all incorporated herein by this reference), and is illustrated by the schematics in FIG. 6 (technique as applied to PAMP) and is described in Example IV.

Briefly, the primers are carried by nanoparticles that are diluted to two nanoparticles per compartment on average through water-in-oil emulsification, to produce droplets containing other PCR reagents and the template nucleic (FIG. 6 (genomic DNA)). Each randomly paired nanoparticle becomes housed within one of the droplets, where correctly paired primers generate millions of copies of the gene fragment, which remain in the droplet. Similar to wells in a microtiter plate, the droplets create a physical barrier to each PCR reaction in them.

The primers containing nanoparticles can be manufactured with various techniques, for example by another warm oil-in-water technique as described in Oyewumi et al. Drug Development and Industrial Pharmacy 28:317-328, 2002. Briefly, each group of primers in solution is mixed into melted emulsifying wax in the presence of an emulsifier, at 55° C. Wax nanoparticles, which average 100 nm in diameter, form when the reaction is cooled to room temperature.

IV. Microarray Analysis of Polymorphisms Identified According to the Assays of the Invention.

For sequence analysis of polynucleotides of interest, genomic tiling microarrays of varying formats have been developed in the art (see, e.g., Liu, Y. T., et al., Clinical Infectious Diseases, San Diego, p. 196, LB-3, “A virus-specific DNA microarray as a diagnostic and discovery tool,” 41^{st} Ann. Meeting of ISDA, 2003; Wang, et al., PNAS USA, 99:15687-15692; 2002; Wang et al. PLoS Biol., 1:E2, 2003; Ishkanian, et al., Natl. Genet., 36:299-303, 2004). Compared to the present cost of direct sequencing, microarray characterization of polynucleotides is relatively cost-effective, and can be readily automated. The use of such arrays is generally illustrated by the schematics in FIGS. 2 and 4, as well as by Example II.

For analysis of boundaries about a deletion, translocation or inversion of one or more nucleotides in a gene, a locus array for the target gene is prepared. For example, a tiling array with an average probe length of 1 kb will cover a 500 kb region. Shorter probes (50-70 bp) should be utilized for analysis of gene sequences with numerous repetitive motifs, such as are found in the CDNK2A/B loci (see, e.g., Bertone, et al. Genome Res., 16:271-281, 2006, incorporated herein by this reference). An assay according to the invention is performed on the array and scanned; e.g., using a commercially available scanner such as the GENEPIX™ 4000B from Axon (see, e.g., Eisen, et al., Methods Enzymol., 303:179-205, 1999). Targeted boundaries are identified in scans as spots with high signals, as illustrated in, for example, FIG. 4.

V. Diagnostic and Therapeutic Monitoring Using Breakpoint-Specific Probes.

Once a deletion segment or corresponding fusion transcript has been characterized, probes can be developed to target them in cells obtained from the same patient. This allows clinicians to practice personalized medicine; e.g. cancer therapy, by monitoring the progression of the patient's cancer (such as by recognizing when the size of a deleted segment is altered or when multiple deletions or translocations occur) or treatment (e.g., if the affected chromosomal region is stabilized).

With knowledge of the boundaries of the sequence variation in hand, the information can be used to diagnose a pre-cancerous condition or existing cancer condition. Further, by quantitating the number of cells in successive cell samples which bear and acquire the deletion or other polymorphism at separate locations in the body and/or over time, the progression of a cancer condition can be monitored. For example, data provided by assaying the patient's tissues at one point in time to detect a first set of sequence variations from wild-type could be compared against data provided from a subsequent assay, to determine if changes in the location, size or number of sequence variations have occurred.

A highly specific adaptation of nested PCR that is particularly preferred technique for quantitating cancer burden with identified signature breakpoint sequences as described in U.S. Pat. No. 5,747,251, the disclosure of which is incorporated herein by this reference and detailed in Example III. Briefly, the technique of the '251 Patent involves competitive PCR is performed using a competitor template containing an induced sequence variation of one or more base pairs which results in the competitor differing in sequence (but not size) from the target template. One of the primers is biotinylated or, preferably, aminated so that one strand (usually the antisense strand) of the resulting PCR product can be immobilized via an amino-carboxyl, amino-amino, biotin-streptavidin or other suitably tight bond to a solid phase support which has been tightly bound to an appropriate reactant.

The bonds between the PCR product, solid phase support and reactant will be covalent ones, thus reliably rendering the bonds resistant to uncoupling under denaturing conditions. Once the aminated or biotinylated strands of the PCR products are immobilized, the unbound complementary strands are separated in an alkaline denaturing wash and removed from the reaction environment. Primers corresponding to the target and competitor nucleic acids are labeled with a detection tag. The primers are then hybridized to the antisense strands in absence of competition from the removed unbound sense strands. Appropriate assay reagents are added and the degree of hybridization is measured by ELISA measurement means appropriate to the detection tag and solid phase support means used, preferably an ELISA microplate reader. The measured values are compared to derive target nucleic acid content, using a standard curve separately derived from PCR reactions amplifying templates including target and competitor templates.

Where a deletion or other polymorphism is found in an individual mammal who has not yet developed symptoms of a disease clinically related to the presence of such deletion or polymorphism, such as cancer, the deletion or polymorphism will be indicative of a genetic susceptibility to develop the cancer condition. Analysis data obtained by performance of the methods of the invention will be of particular prognostic value where the abnormality is carried in germline cells and/or has the individual has a family history of a particular cancer condition.

Where other indicia of the presence of the disease in the individual are present, such as clinical symptoms, biopsy results, positive radiological examinations or the like, analysis data obtained by performance of the methods of the invention indicating the presence of a deletion or translocation of one or more nucleotides in genomic DNA clinically related to the occurrence of the disease will also be of particular diagnostic value.

A determination of susceptibility to disease or diagnosis of its presence can further be evaluated on a qualitative basis based on information concerning the prevalence, if any, of the cancer condition in the patient's family history and the presence of other risk factors, such as exposure to environmental factors and whether the patient's cells also carry a deletion of another gene; e.g., for both CDKN2A and MTAP, as occurs in many primary cancers. Multiple gene deletions and translocations of the kind that occur in connection with the CDKN2A and MTAP coding sequences are of particular diagnostic and cancer monitoring utility.

For example, as described in U.S. Pat. No. 6,689,561 (the disclosure of which is incorporated herein by this reference), screening of cells from a known leukemia cell line (U937; ATCC Accession No. 1593) indicates that they contain an intragenic microdeletion of 18 base pairs in the CDK4I5′ exon (see, '561 Patent at Example VI). Using such information and the techniques for identifying sequence variations in genes which are illustrated herein, those of ordinary skill in the art will be able to screen cell samples from particular 9p21-linked tumors for reproducible polymorphisms and/or deletions of CDK4I to determine genetic susceptibility to, as well as the existence of a cancer condition as defined herein (particularly melanomas, gliomas, non-small cell lung cancers and leukemias).

TMPRSS2:ETS gene fusions are also a recurrent prostate cancer-specific event. Among all of the reported fusion partners in the ETS family of genes, TMPRSS2:ERG is the most prevalent one, variants of which have been associated with progressive diseases. As described in Examples VII and VIII, TMPRSS2:ERG fusion transcripts were detected with total RNA from 3 cells containing the fusion in the presence of more than 3000 times excess of background RNA and in a primary prostate tumor having no more than 1% of cancer cells. The ability to detect multiple transcript variants is critically dependent on both the primer and probe designs. The methods of the invention will therefore facilitate clinical studies of transcript RNA and can be readily adapted to include other fusion genes.

The invention having been fully described, aspects of its practice are illustrated by the examples below. The scope of the invention shall not, however, be limited by the examples, but is instead defined by the appended claims on issuance of this application, or any applications which claim the priority of this application. Standard abbreviations are used in the examples, such as “ml” for milliliters, “min” for minutes, and the like.

### Example I

### Representative Target Gene Deletions and Primer Pair Construction

The invention may be applied to any gene or other polynucleotide in which at least a portion of the primary sequence outlying a potential deletion, translocation or inversion region is known, so appropriate primer pairs can be developed to hydridize to the target molecule at loci a distance apart; e.g., ≧1 kb apart for application of the PAMP method, or at loci <1 kb apart for the PAMP/poison primer embodiment of the invention. For purposes of illustrating application of the invention to genomic DNA, the examples herein are of use of the invention to characterize deletions in the CDKN2 region on human chromosome 9p21. The CDKN2 region experiences homozygous deletions in a diverse range of cancer cell lines, and so is a exemplary target molecule to demonstrate the use and sensitivity of the invention. However, those of ordinary skill in the art will understand that application of the invention is not limited to the CDKN2 region of human chromosome 9p21, or to any particular chromosome or polynucleotide, or to genomic DNA of any particular species.

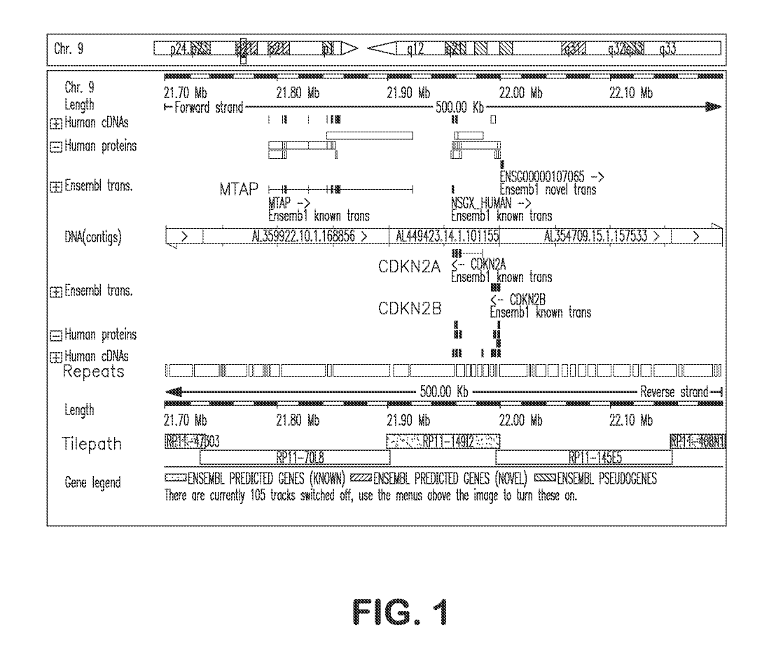

The CDKN2 region on human chromosome 9p21 encodes three different tumor suppressor genes (FIG. 1). The p16^{INK4a} (one of the CDKN2A products) and p15^{INK4b} (CDKN2B product) proteins constrain cell cycle progression by the Rb pathway. The p14^{ARF} (the other alternative reading frame of CDKN2A) gene product regulates the expression of MDM2, the turnover of p53, and thereby controls the cellular response to stress. Because the Rb and p53 pathways are central to cancer gate-keeping and caretaking, strong selection pressures exist for the disruption of the entire CDKN2A gene segment on both chromosomes.

Homozygous deletions of chromosome 9p encompassing the CDKN2A region are very common in cancer cell lines of diverse origin, including lines derived from tumors of the lung, bladder, brain, head and neck, ovary, pancreas, skin, and blood, a finding later confirmed in many types of primary tumors. In addition, CDKN2A inactivation reportedly happens early during cancer development in some well documented solid tumors, including pancreatic adenocarcinoma, head and neck squamous cell cancer and esophageal cancer.

To cover a 500 kb genomic sequence on chromosome 9p21 flanking the CDKN2A locus, 500 primary primers (250 pairs) were synthesized and divided into 20 groups, each with about 25 primers. 10 primer groups were forward (F1-F10) and 10 were reverse (R1-R10), as shown in FIG. 3.

For secondary amplification (to increase the specificity of the PCR reactions and further amplify the PCR products from multiplex PCR reactions performed with the primary primers), secondary groups of primers were developed.

Primer sets can be selected with Primer3 (http://frodo.wi.mit.edu/cgi-bin/primer3/primer3_www.cgi) or GeneRunner (http://www.generunner.com/) avoiding repetitive sequences, which are predicted by Repeatmasker (http://www.repeatmasker.org/cgi-bin/WEBRepeatMasker). The specificities of the PCR primer pairs may also be evaluated by in silico PCR (http://www.genome.ucsc.edu/cgi-bin/hgPcr?command+start). Alternatively, primer design may be assisted by the optimization technique described in co-pending, commonly assigned U.S. Provisional Patent Application No. 60/931,793, filed Mar. 25, 2007.

### Example II

### Deletion Breakpoint Cloning by PAMP

A simplified PAMP scheme is shown in FIG. 2. The series of primers synthesized were toward INK4A exon 1-2 along the CDKN2A locus. Coarse mapping had previously indicated that the Detroit 562 cell line had an approximate 20 kb deletion in a region of rich in repetitive sequences (Nobori, et al. Nature, 368:753-756, 1994).

Groups of forward and reverse primers (generated as described in Example I) were used to generate amplicons from 100 ng of genomic DNA templates for multiplex PCR (conditions: 35 cycles of 92° C., 30 seconds; 55° C., 2.5 minutes). The products were subsequently used as templates for another round of amplification with the same PCR protocol except replacing dTTP by a 4:1 mixture of aminoallyl dUTP (Ambion, Austin, Tex.) and dTTP for probe labeling.

For ease of analysis, an INK4A exon 1-2 minigenomic tiling array was created to cover a 25 kb fragment in the CDKN2A locus (see, FIG. 4A). The labeled amplicons were purified and coupled with Cy3 or Cy5 esters (GE Healthcare, Piscataway, N.J.), purified and hybridized to arrays at 63° C. overnight essentially as previously described in Eisen, et al., Methods Enzymol., 303:179-206 (1999); and Wang, et al., PLoS Biol., 1:E2 (2003). The hybridized arrays were washed and scanned with GenePix 4000B scanner (Molecular Devices, Sunnyvale, Calif.).

For the array, DNA probes were generated by PCR on non-repetitive genomic sequences with BAC clone RP11-14912 (obtained from BACPAC Resources Center at Children's Hospital Oakland Research Institute, Oakland, Calif.). The template probes were printed on poly-L-lysine slides at 0.1 mg/ml. Human Cot-1 DNA (Invitrogen, Carlsbad, Calif.), which is enriched for repetitive sequences, and herring sperm DNA (Promega, Madison, Wis.), which was used as nonspecific control, were also spotted on the array as described by the manual of the commercially available DeRisi™ arrayer.

Only spots with probes close to the breakpoints hybridized to the amplicons when Detroit 562 genomic DNA was used as a template (FIG. 4B). Almost no signal was detected when HEK293 genomic DNA was used as a template. Interestingly, the control HEK293 sample had a significantly higher signal on Cot-1 DNA spots, suggesting that labeled non-exponentially amplified products could produce only weak signals on the tiling probes (the first row in FIG. 4B) due to repetitive sequences downstream of the primers.

In addition, four separate arrays were used to hybridize the individual PAMP products described above. Only F2-R1 produced the same result as shown in FIG. 4B. In contrast, the other three pairs yielded only faint background signals on the arrays. This result indicates that PAMP product pooling with a single array analysis gives the same breakpoint information as four individual arrays. The data support the original experimental predictions, and suggests that the procedure should be generally applicable for deletion and translocation scanning.

Secondary PCR was performed using the nested PCR with pairs of specific primers designed according to the earlier PAMP results. The PCR product was labeled for array hybridization, yielding a result very similar to that shown in FIG. 4B. A simple plot of signal intensity ratio of variant/WT PCR products on the tiling array revealed the genomic location of the breakpoint (FIG. 5A). This analysis shows a very straightforward readout—the location of the deletion is bordered by two peaks. Furthermore, the single major product of the PCR reactions was resolved by agarose gel electrophoresis, excised, extracted and sequenced (FIG. 5B-5D).

To mimic the heterogenous population of cancer and host cells typically found in solid tumors, various genomic DNA ratios of Detroit 562 (variant) and HEK293 (wild type) were used as templates for PAMP and array hybridization. CDKN2A deletion was detected when only 2% of variant genomic DNA was present in these experiments. The array result with contaminated DNA was as clean as in FIGS. 4B and 5A. We also used the same primers described for the aforementioned nested PCR, which produced the bands shown in FIGS. 5B and 5C.

The same approach was used to map the breakpoint of a breast cancer cell line (Hs 578T), quickly yielding a result consistent with another report (Sasaki, et al., Oncogene, 22:3792-3798, 2003).

### Example III

### Alternative Method for Secondary PCR to Increase the Specificity of the Results From the Primary Multiplex PCR for Deletion Breakpoints

The solid phase capture PCR methodologies described in U.S. Pat. No. 5,747,251 are particularly useful alternatives to standard nested PCR techniques as described in Example II. The disclosure of the patent is incorporated by reference herein; briefly, the methods described are summarized as follows:

A. Performance of Method.

Conventional or anchored PCR is performed to coamplify the target and competitor templates using the modified and unmodified primers. The PCR products may be purified by minicolumn (using, for example, the MAGIC PCR PREPS™ product from Promega, Madison, Wis.). The resulting products will consist of antisense strands having the coupling agent attached thereto and sense strands without coupling agent.

Immobilization or capture of antisense strands is performed by placing a diluted aliquot of the double-stranded PCR products onto the solid phase support (e.g., coated ELISA plate wells). The PCR products are allowed to stand in the plate wells in the presence of a coupling reagent for a period of time sufficient for capture of the antisense strands bearing the coupling agent to the reagent coating each well. Sense strands are then separated from the captured antisense strands and removed from the solution in each plate well by incubation with an alkaline denaturing salt (such as 0.1N NaOH) and washing with a buffer solution.

After removal of the unbound sense strands, the labeled probes are added to each plate well and hybridization is allowed to occur with the captured antisense strands. A substrate, antibody or other assay reagent appropriate to interact with the label used on the probes is added to each plate well and the reaction stopped at an appropriate point. An ELISA microplate reader (such as the THERMO MAX™ microplate reader from Molecular Devices of Menlo Park, Calif.) is used to measure absorbance in each well and the values compared to a standard curve to derive input DNA content.

If a chemiluminescent hybridization detection tag is used and a reagent, such as an alkaline phosphatase substrate, is added to react with the detection tag, emitted photons will be measured instead of OD. This approach enhances the linear range of the measurements in that it avoids the loss of sensitivity in OD measurements experienced at high OD values. A suitable microplate reader for use with a chemiluminescent tag is commercially available from Dynatech of Chantilly, Va. Where a fluorescent tag is used, a suitable ELISA microplate reader is commercially available from Millipore of Boston, Mass.

B. Generation of Standard Curves for Calculation of the Ratio of Target to Competitor Nucleic Acid

Probe hybridization-based quantification of PCR products can eliminate false positive results derived from non-specific amplification. However, potential flaws can come from differences in hybridization or labeling efficiency of the probes. An exemplary construct for this purpose has tandemly arranged wild type DNA and competitor DNA sequences. Since the standard curves are generated from the results of hybridization of each probe with the standard construct, labeling or hybridization efficiency does not affect the results.

A nucleic acid standard (hereafter referred to for convenience as “standard DNA” construct) is constructed according to means known in the art to include two tandemly aligned DNA regions from wild type (target) DNA and competitor DNA. Conventional PCR is performed to amplify the standard DNA using a reactant modified primer (such as an aminated primer) and a regular primer. Two sets of serial dilutions of the standard DNA PCR products are prepared.

Separately, samples containing target DNA to which competitor DNA was added in known quantity are coamplified. Two aliquots of the PCR products are made and added to microtiter plate wells for covalent coupling of antisense strands and removal of sense strands. Hybridization is performed separately with the two sets of standard DNA solutions and the two sample/competitor DNA aliquots. The SSO's correspond to the target sequence and the competitor sequence. After hybridization, an assay appropriate to the detection tag used is performed and optical density or another appropriate value is measured with appropriate hybridization detection means.

Where the SSO's are not known to have equal hybridization efficiencies, a separate standard curve is generated for each SSO based on the adsorbence (OD) readings provided by use of the microtiter plate reader. (Only one curve is needed where no differences in the hybridization efficiencies for each probe is expected). ELISA data analysis software (such as the DELTA SOFT version 2.1 sold by BioMetallics of Princeton, N.J.) is then used to calculate the amount of target DNA in the sample. Using this data, a ratio of target to competitor products can be calculated.

Where the approximate amount of target DNA present in the sample is not known, target DNA samples can be mixed with various known concentrations (usually three) of competitor DNA. Competitive PCR is then performed according to the methods described herein and the ratio of target DNA to competitor calculated. A graph is then generated which plots the known concentrations of competitor against the ratios of target and competitor sequences determined by covalent capture PCR in logarithmic scales. Calculation of the amount of competitor which would give a 1:1 ratio will provide the approximate concentration of target DNA in the starting samples.

### Example IV

### Water-in-Oil Emulsion PCR Protocol

The following protocol is adapted from Diehl, et al., supra, and can be adapted for use with available equipment and reagents by those of ordinary skill in the art.

Prepare emulsifier-oil mix using 7% (wt/vol) ABIL WE09™, 20% (vol/vol) mineral oil and 73% (vol/vol) Tegosoft™ DEC. Vortex this mix briefly and incubate at 18-25° C. for 30 min. Store the mixture at room temperature for no longer than 2 d. Dilute the template DNA with TE to ˜20 μM immediately before use. DNA at low concentrations can stick to tubes during storage.

Set up a 150 μl amplification reaction by mixing the following: Primer 5 (2.5 μM) 3 μl, Primer 6 (400 μM) 3 μl, Template DNA (˜20 μM) 10 μl, Beads 6 μl, dNTPs mix 3 μl, 10×PCR buffer 15 μl, Platinum Taq DNA polymerase (5 U/μl) 9 μl, Water 101 μl.

Add, in order, one steel bead, 600 μl oil-emulsifier mix (Step 9) and 150 μl PCR mix to one well of a 96-well storage plate. Seal plate with adhesive film. Turn the plate upside down to make sure the steel bead moves freely in the well. Avoid excess oil on the rims of the wells as the adhesive film will not seal.

Purchase or assemble a TissueLyser™ adaptor set by sandwiching the 96-well storage plate containing the emulsion PCR mix between the top and bottom adapter plates, each fitted with a compression pad facing the 96-well storage plate. Place the assembly into the TissueLyser holder, and close the handles tightly. When using less than 192 wells, balance TissueLyser with a second adaptor set of the same weight. Mix once for 10 s at 15 Hz and once for 7 s at 17 Hz.

Disassemble the adaptor set and centrifuge the plate for 10 s at ˜3 g to get the liquid to the bottom. Assess the quality of the emulsions at 400× magnification with an inverted microscope. Dip a pipette tip into the emulsion, and streak it over the bottom of a 48-well cell culture plate. Aliquot 80 μl of the emulsion into eight wells of a 96 well PCR plate. Centrifuge the plate for 10 s at ˜3 g to get the liquid to the bottom.

Pipette emulsions slowly to avoid shear force. Temperature cycle the emulsions according to the following program: 2 min at 94° C., 2-4 15 s at 98° C., 45 s at 64° C., 75 s at 72° C., 15 s at 98° C., 45 s at 61° C., 75 s at 72° C., 15 s at 98° C., 45 s at 58° C., 75 s at 72° C., 15 s at 98° C., 45 s at 57° C., and 75 s at 72° C.

To each 80 μl emulsion, add 150 μl of Breaking buffer and pipette up and down three times to mix. Seal the PCR plate, place it into an empty 96-well storage plate, and assemble this between two TissueLyser™ adaptor plates as described. Place in TissueLyser™ and mix for 30 s at 20 Hz. Remove PCR plate from the TissueLyser and centrifuge for 2 min at 3,200 g. Remove the top oil layer with a 20 μl pipette tip attached to a vacuum manifold. Add 150 μl of Breaking buffer, seal the plate and centrifuge again for 2 min at 3,200 g. Place the plate in a 96-well magnetic separator for 1 min, and completely remove the liquid with a pipette.

Remove the plate from the magnet, resuspend the beads in 100 μl of TK buffer and pool the beads from the eight wells into a 1.5 ml tube. Place the tube on the magnet to concentrate the beads for 1 min, and carefully remove the supernatant with a pipette tip. This removes the non-biotinylated DNA strand from the beads. Resuspend the beads in 100 μl of TK buffer. The recovery of beads can be assessed by measuring absorption at 600 nm. A spectrophotometer is convenient for this purpose. An aliquot of the beads coated with Primer 5 can be used as a fiducial. The typical recovery with the procedure described is 50-70%.

Set up the oligohybridization in a 96-well PCR plate by mixing the following: Primer 3 (1 μM) 10 μl, Beads 20 μl, 5× hybridization buffer 20 μl, Water 50 μl.

The amount of beads to be used depends on the nature of the experiment. Ten million beads provide a great enough mass to be seen during magnetic collection and facilitate recovery. The recovery can be assessed by measuring absorption at 600 nm as described above.

To break emulsions and detect DNA on the beads, incubate the reaction at 50° C. for 15 min in a thermal a cycler. Place the plate on a 96-well magnetic separator for 1 min to concentrate the beads, and remove 80 μl of the supernatant with a pipette. Wash beads twice with 80 μl of TK buffer. Use flow cytometry to determine the relative fluorescence intensity of the primers hybridized to the DNA on the beads. Alternatively, fluorescence microscopy can provide a rapid qualitative analysis of the beads generated. Empirically, establish the amplifier gain (voltages) for the detection of the forward scatter and side scatter.

### Example V

### Poison Primer Amplification of Deleted Region in Target Polynucleotide

Once the primary multiplex PCR of the invention has identified deletion breakpoints in the target polynucleotide, it is possible to design primers to target the deleted sequence in the wild-type molecule. A pair of primers external to the deletion segment and a primer (“poison” primer) internal to it are utilized (see, schematic in FIG. 7).

As described in Edgley, et al., Nuc. Acids Research, 30:e52, 2002, amplification from the wild type template produces two fragments, one full length and the other shorter. The latter will generally be produced more efficiently. Amplification from the variant template (lacking the deletion segment) produces a single variant fragment from the external primers. In a further PCR reaction, a primer pair is placed just inside the external first round primers.

The shorter wild-type band from the first round can't serve as a template for the second round because it lacks a primer binding site. The longer wild-type fragment does serve as a template but, because its production was limited in the first round of PCR, its production in the second round is also limited; i.e., was “poisoned” by the primer corresponding to the deleted segment.

### Example VI

### Sensitivity of the Method of the Invention for Deletion Segments in Genomic DNA in the Presence of an Excess of Wt Genomic DNA

The PAMP method was performed as described in Examples I and II. The total genomic DNA for each assay was 100 ng. This is equivalent to about 28000 copies of haploid genome (based on the estimate of 2.8×10⁵ molecules/ug of haploid genome). The CDKN2A deleted cell line Detroit 562 was serially diluted with CDKN2A wild type HEK293 as shown in Table 1, below.

| Complexity | Absolute genome copy | Array |
|---|---|---|
| Detroit 562:Total | number of Detroit 562 | Result |
| 1:1 | 28000 | P |
| 1:10 | 2800 | P |
| 1:50 | 560 | P |
| 1:100 | 280 | P |
| 1:200 | 140 | P |
| 1:600 | 47 | P |
| 1:1800 | 16 | P |
| 1:5400 | 5 | N |
| 0:1 | 0 | N |

The assay was able to detect about 1 breakpoint sequence in the presence of 2000 fold excess of wild type genome with sensitivity of detecting 5-16 such molecules. Thus, the invention provides a method for detecting genomic DNA deletions in the presence of an excess of wild-type DNA; e.g., more than 99.9% wild-type.

### Example VII

### Microassay-Based TMPRSS2:ERG Exon Fusion Mapping

Most of the TMPRSS2:ERG fusion junctions are between exons 1 or 2 of the TMPRSS2 and exons 2-5 of the ERG. Such constraints perhaps are related to whether a functional ERG protein can be made from the gene fusions. Therefore, a pair of primers at exon 1 of the TMPRSS and exon 6 of the ERG for RT-PCR were used.

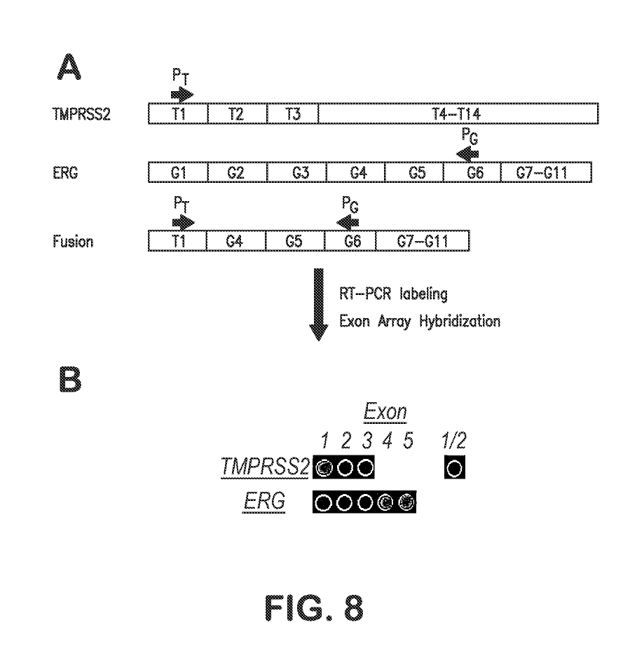

As shown in FIG. 8A, PCR products were only generated when there was a gene fusion, since the two primers are located at different genes. Subsequently, the PCR products were labeled and hybridized to an exon array for mapping the exons near the fusion junction. Printed on the array are 30 mer oligonucleotide probes derived from exons 1-3 of the TMPRSS2 and exons 1-5 of the ERG (see Table 2, below). Each selected sequence is represented by two complementary probes (F: forward and R: reverse complement) since sometimes PCR labeled amplicons may bind to only one strand of the probe based empirical observation.

TABLE 2

| Name | Sequence |
|---|---|
| T1F | GGGCGGGGAGCGCCGCCTGGAGCGCGGCAG |
| T2F | ACATTCCAGATACCTATCATTACTCGATGC |
| T3F | GGTCACCACCAGCTATTGGACCTTACTATG |
| T1/2F | TGGAGCGCGGCAGGTCATATTGAACATTCC |
| G1F | AGGGACATGAGAGAAGAGGAGCGGCGCTCA |
| G2F | AGACCCGAGGAAAGCCGTGTTGACCAAAAG |
| G3F | GCTGGTAGATGGGCTGGCTTACTGAAGGAC |
| G4F | TTATCAGTTGTGAGTGAGGACCAGTCGTTG |
| G5F | CTCTCCTGATGAATGCAGTGTGGCCAAAGG |
| T1R | CTGCCGCGCTCCAGGCGGCGCTCCCCGCCC |
| T2R | GCATCGAGTAATGATAGGTATCTGGAATGT |
| T3R | CATAGTAAGGTCCAATAGCTGGTGGTGACC |
| T1/2R | GGAATGTTCAATATGACCTGCCGCGCTCCA |
| G1R | TGAGCGCCGCTCCTCTTCTCTCATGTCCCT |
| G2R | CTTTTGGTCAACACGGCTTTCCTCGGGTCT |
| G3R | GTCCTTCAGTAAGCCAGCCCATCTACCAGC |
| G4R | CAACGACTGGTCCTCACTCACAACTGATAA |
| G5R | CCTTTGGCCACACTGCATTCATCAGGAGAG |

T: TMPRSS2; G: ERG
F: forward probe; R: reverse complement probe

A prostate cancer cell line, VCaP, with a TMPRSS and ERG fusion was used for initial feasibility testing. The RT-PCR reaction was performed with an OneStep RT-PCR kit (Qiagen, Valencia, Calif.) essentially following the manufacturer's protocol, except that the final reaction volume was scaled down to 20 μl. The forward (GTT TCC CAG TCA CGA TCC AGG AGG CGG AGG GGG A) and reverse primers (GTT TCC CAG TCA CGA TCG GCG TTG TAG CTG GGG GTG AG) are located at exon 6 of ERG and exon 1 of TMPRSS2 respectively. The 5′ ends of both primer have the sequence of primer B (GTT TCC CAG TCA CGA TC) for the subsequent step of PCR labeling with a single primer B.

Briefly, the RT-PCR reaction was assembled at 4° C. in a PCR workstation and transferred to a thermocycler with the block preheated to 50° C. for 30 minutes and followed by 95° C. for 15 minutes to activate HotStar™ Taq DNA polymerase as well as to inactivate the reverse transcriptases. The PCR conditions were 35 cycles at 92° C. for 30 seconds, 55° C. for 30 seconds and 68° C. for 1.5 minutes with a final extension step at 68° C. for 5 minutes. One μl of unpurified product was subsequently used as a template for another 20 cycles of amplification to label the amplicons via a “Round C” PCR protocol (94° C. for 30 seconds, 40° C. for 30 seconds, 50° C. for seconds and 72° C. for 1 minute) with primer B and 4:1 mixture of aminoallyl dUTP (Ambion, Austin, Tex.) and dTTP for probe labeling (20). The labeled amplicons were purified with DNA Clean-up and Concentrator-5 columns (Zymo Research, CA). eluted in 9 μl of sodium bicarbonate (pH 9.0) and couple with 1 μl of DMSO dissolved Cy3 NHS esters (GE Healthcare, Piscataway, N.J.) for 30 to 60 minutes. The Cy3 labeled amplicons were purified with DNA Clean-up and Concentrator-5 columns and eluted with 10 μl of 10 mM Tris-HCL (pH 8.0). Then, the Cy3 labeled amplicons were diluted in water and combined with 3.6 μl of 20×SSC, 0.5 μl of Hepes (pH 7.0) and finally 0.5 μl of 10% SDS to reach final volume of 25 μl.

The total RNA was subjected to RT-PCR with a pair of primers located at exon 6 of ERG and exon 1 of TMPRSS2. The unpurified product was labeled and hybridized on the microarray (FIG. 8B). Only spots corresponding to exon 1 of TMPRSS2 and exons 4-5 of ERG develop strong signals. This results indicates the fusion junction is at the exon 1 of TMPRSS2 and exon 4 or ERG.

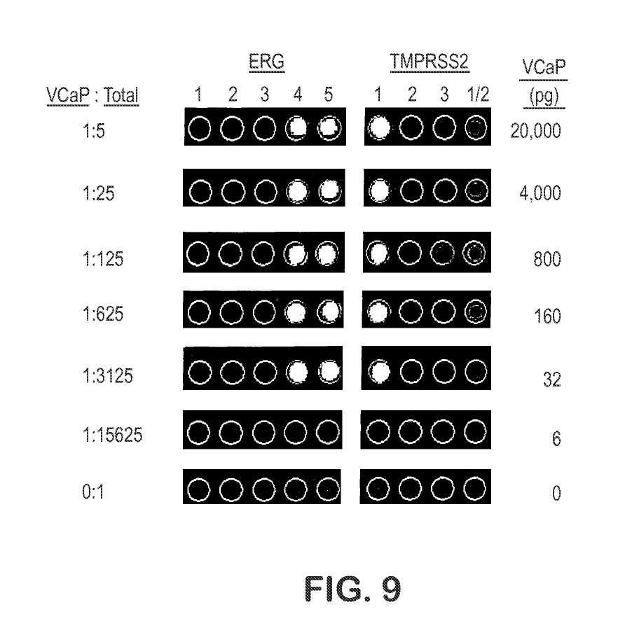

To mimic a typical clinical situation, in which small population of cancer cells are present among normal host cells in a primary tumor, decreasing amounts of total RNA extracted from VCaP cells were spiked into an excess of HeLa RNA, which does not have the fusion transcripts. The detection limit reached by this particular assay was 32 pg of VCaP RNA in the presence of 100 ng of HeLa RNA (FIG. 9). This translates into only 1-3 cancer cells in the presence of 3000 times more normal cells.

To test the ability of the exon mapping array to detect and characterize TMPRSS2:ERG fusion transcripts in clinical samples, total RNA was isolated from frozen unpurified primary prostate tissues obtained during surgery. Many of these tumors had a substantial fraction of normal stromal cells. Total RNA (5-50 ng) from prostate cancers (n=20) and non-malignant hyperplastic prostate tissues (n=10) were subjected to RT-PCR labeling and array hybridization. The results showed that 7 of 20 cancer but zero of 10 non-malignant samples had TMPRSS2:ERG fusion genes.

To confirm the presence of the gene fusion, direct sequencing was performed for the 7 TMPRSS2:ERG positive samples. The sequencing data validated the exon fusion findings revealed by the array assay. Some samples clearly showed two or more bands on the agarose gel when the PCR products were subjected to electrophoresis, corresponding to two or more fusion transcripts in the same specimens. Therefore, multiple fusion transcripts may be detected in a single sample. The results are shown in Table 3:

| | | Gleason | |
|---|---|---|---|
| Sample # | cancer % | grade | Fusion trancsripts |
| 1 | 30 | 7 | T1-G4:T2-G4 |
| 2 | 20 | 5 | |
| 3 | 50 | 5 | |
| 4 | 20 | 6 | T1-G4 |
| 5 | 80 | 9 | |
| 6 | 1 | 6 | |
| 7 | 90 | 8 | |
| 8 | 20 | 4 | |
| 9 | 80 | 8 | |
| 10 | 1 | 6 | T1-G2 |
| 11 | 2 | 6 | |
| 12 | 70 | 7 | |
| 13 | 20 | 9 | T1-G4 |
| 14 | 1 | 6 | |
| 15 | 70 | 8 | T1-G4:T2-G4 |
| 16 | 20 | 8 | |
| 17 | 80 | 8 | T1-G4:T2-G4 |
| 18 | 50 | 7 | T1-G2, T1-G3, T1-G4 |
| 19 | 80 | 7 | |
| 20 | 80 | 7 | |

Consistent with the VCaP titration study (FIG. 9), the clinical assay can detect the fusion transcript when only 1% of the cells in the prostate tissue sample are tumor cells (Sample 10).

The invention having been fully described, modifications, equivalents and extensions thereof may become obvious to those of skill in the art in view of this disclosure. All such modifications, equivalents and extensions are considered to be within the scope of the invention and appended claims.

What is claimed is:

1. A method for detecting a variant polynucleotide having a nucleotide sequence differing from the wild-type nucleotide sequence of a nucleic acid molecule, wherein the variant polynucleotide is in a sample containing up to about 99.9% of the wild-type nucleic acid molecule, the method comprising:
a. a primary amplification round of multiplex PCR with a multiplicity of primer pairs designed to hybridize to loci on the wild-type nucleic acid molecule approximately evenly spaced around the locus of interest; and,
b. analysis of the sequence of any variation identified in step (a).

2. The method according to claim 1, wherein the nucleic acid molecule is DNA.

3. The method according to claim 2, wherein the nucleic acid molecule is genomic DNA.

4. The method according to claim 3, wherein the loci for hybridization of the primer pairs are spaced ≧1 kb apart.

5. The method according to claim 1, further comprising a step (c) consisting of a secondary amplification round of nested PCR using at least three primers, wherein two of the primers flank the boundaries of any variation identified in step (b) and the third primer hybridizes to the wild-type genomic DNA sequence.

6. The method according to claim 5, wherein the nested PCR in step (b) utilizes a poison primer.

7. The method according to claim 6, wherein the loci for primer hybridization are spaced less than 1 kb apart.

8. The method according to claim 1, wherein the nucleotide sequence variation is a deletion, translocation or inversion of one or more nucleotides.

9. A method for detecting a deletion, translocation or inversion of one or more nucleotides in genomic DNA, wherein the genomic DNA containing the deletion or translocation is present in a sample containing an excess of the wild-type genomic DNA, the method comprising:
a. a primary amplification round of multiplex PCR with a multiplicity of primer pairs designed to hybridize to loci on the wild-type genomic DNA molecule spaced approximately evenly apart around the locus of interest;
b. a secondary amplification round of nested PCR using at least three primers, wherein two of the primers flank the boundaries of any variation identified in step (a) and the third primer hybridizes to the wild-type genomic DNA sequence; and,
c. analysis of the sequence of any variation segment identified in steps (a) and (b).

10. The method according to claim 9, wherein greater than 50% of the genomic DNA present in the sample has the wild-type nucleotide sequence.

11. The method according to claim 9, wherein about 99.9% of the genomic DNA present in the sample has the wild-type nucleotide sequence.

12. The method according to claim 9, wherein the loci for primer hybridization are spaced ≧1 kb apart.

13. The method according to claim 1, further comprising step (a)′ wherein the nucleic acid molecule is pre-screened for the presence of variations from wild-type prior to the primary amplification step.

14. The method according to claim 9, wherein the nested PCR in step (b) utilizes a poison primer.

15. The method according to claim 14, wherein the loci for primer hybridization are spaced less than 1 kb apart.

16. The method according to claim 1, wherein the analysis in step (b) is performed by sequencing on a genomic tiling array.

17. The method according to claim 1, wherein the analysis in step (b) is performed by water-in-oil PCR.

18. The method according to claim 1, wherein the analysis in step (b) is performed by direct sequencing.

19. The method according to claim 9, wherein the analysis in step (c) is performed by sequencing on a genomic tiling array.

20. The method according to claim 9, wherein the analysis in step (c) is performed by water-in-oil PCR.

21. The method according to claim 9, wherein the analysis in step (c) is performed by direct sequencing.

22. The method according to claim 1, wherein the primer pairs utilized in step (a) are enclosed in nanoparticles and the nanoparticles are randomly assembled into droplets along with reagents for PCR in a single tube.

23. Isolated polynucleotides hybridizable to the boundaries of a deletion, translocation or inversion in genomic DNA, wherein the boundaries were identified according to the method of claim 1.

24. A method for diagnosis of a disease clinically related to the occurrence of a deletion or translocation of one or more nucleotides in genomic DNA from an individual, the method comprising correlation of the analysis data obtained by performance of the method of claim 1 to clinically acceptable indicia of the presence of the disease or disorder in the individual.

25. The method according to claim 24, wherein the disease is cancer.

26. A method for monitoring the progression of cancer in a patient, wherein the progression is clinically related to changes in the location, size or number of deletions, translocations or inversion's in genomic DNA, the method comprising determining whether the location, size or number of targeted deletions, translocations or inversions in an individual's cancer cells is altered over time or as between different cell populations sampled from the patient, wherein the determining is performed by assaying the genomic DNA contained in the cells using polynucleotides according to claim 23.

27. The method according to claim 26, wherein the assaying of the genomic DNA is performed according to the method of claim 1.

28. The method according to claim 26, wherein the determining step is performed by competitive PCR.

29. A method for monitoring the progression of cancer in a patient, wherein the progression is clinically related to the presence of an RNA fusion transcript in sample of the individual's cancer cells, wherein the RNA fusion transcript corresponds to the presence of a deletion or translocation in genomic DNA previously identified according to claim 1, the method comprising determining whether the concentration or number of such RNA fusion transcripts is altered over time or as between different cell populations sampled from the patient.


SEQUENCE LISTING (22 sequences)

SEQ ID NO: 1 - DNA, 30 bp, Artificial Sequence
  PCR Probe
gggcggggagcgccgcctggagcgcggcag

SEQ ID NO: 2 - DNA, 30 bp, Artificial Sequence
  PCR Probe
acattccagatacctatcattactcgatgc

SEQ ID NO: 3 - DNA, 30 bp, Artificial Sequence
  PCR Probe
ggtcaccaccagctattggaccttactatg

SEQ ID NO: 4 - DNA, 30 bp, Artificial Sequence
  PCR Probe
tggagcgcggcaggtcatattgaacattcc

SEQ ID NO: 5 - DNA, 30 bp, Artificial Sequence
  PCR Probe
agggacatgagagaagaggagcggcgctca

SEQ ID NO: 6 - DNA, 30 bp, Artificial Sequence
  PCR Probe
agacccgaggaaagccgtgttgaccaaaag

SEQ ID NO: 7 - DNA, 30 bp, Artificial Sequence
  PCR Probe
gctggtagatgggctggcttactgaaggac

SEQ ID NO: 8 - DNA, 30 bp, Artificial Sequence
  PCR Probe
ttatcagttgtgagtgaggaccagtcgttg

SEQ ID NO: 9 - DNA, 30 bp, Artificial Sequence
  PCR Probe
ctctcctgatgaatgcagtgtggccaaagg

SEQ ID NO: 10 - DNA, 30 bp, Artificial Sequence
  PCR Probe
ctgccgcgctccaggcggcgctccccgccc

SEQ ID NO: 11 - DNA, 30 bp, Artificial Sequence
  PCR Probe
gcatcgagtaatgataggtatctggaatgt

SEQ ID NO: 12 - DNA, 30 bp, Artificial Sequence
  PCR Probe
catagtaaggtccaatagctggtggtgacc

SEQ ID NO: 13 - DNA, 30 bp, Artificial Sequence
  PCR Probe
ggaatgttcaatatgacctgccgcgctcca

SEQ ID NO: 14 - DNA, 30 bp, Artificial Sequence
  PCR Probe
tgagcgccgctcctcttctctcatgtccct

SEQ ID NO: 15 - DNA, 30 bp, Artificial Sequence
  PCR Probe
cttttggtcaacacggctttcctcgggtct

SEQ ID NO: 16 - DNA, 30 bp, Artificial Sequence
  PCR Probe
gtccttcagtaagccagcccatctaccagc

SEQ ID NO: 17 - DNA, 30 bp, Artificial Sequence
  PCR Probe
caacgactggtcctcactcacaactgataa

SEQ ID NO: 18 - DNA, 30 bp, Artificial Sequence
  PCR Probe
cctttggccacactgcattcatcaggagag

SEQ ID NO: 19 - DNA, 34 bp, Artificial Sequence
  Primer
gtttcccagtcacgatccaggaggcggaggggga

SEQ ID NO: 20 - DNA, 38 bp, Artificial Sequence
  Primer
gtttcccagtcacgatcggcgttgtagctgggggtgag

SEQ ID NO: 21 - DNA, 17 bp, Artificial Sequence
  Primer
gtttcccagtcacgatc

SEQ ID NO: 22 - DNA, 55 bp, Homo sapiens
gcgcgtgagctgaggcaagaccggagactgtatgtttactctgtactgatttaaa